(19) United States
(12) Patent Application Publication
(10) Pub. No.: US 2020/0069872 A1
(43) Pub. Date: Mar. 5, 2020

(54) PIERCING MECHANISM AND CARTRIDGE PIERCING SYSTEM

(71) Applicant: Novartis AG, Basel (CH)

(72) Inventors: Alain Schmidlin, Basel (CH); Mario Iobbi, Basel (CH); Erich Studer, Basel (CH); Andrew Bryant, Basel (CH); Chinmay Deodhar, Pune (IN); Rajan Patel, Menlo Park, CA (US)

(21) Appl. No.: 16/493,740

(22) PCT Filed: Mar. 14, 2018
(86) PCT No.: PCT/EP2018/056368

§ 371 Date: Sep. 12, 2019

Related U.S. Application Data
(60) Provisional application No. 62/471,990, filed on Mar. 16, 2017.

(30) Foreign Application Priority Data
Mar. 31, 2017 (EP) 17164098.0

(51) Int. Cl. A61M 5/162; A61M 5/145; A61M 5/315; A61M 5/50

(52) U.S. Cl. CPC A61M 5/162; A61M 5/14566; A61M 2005/2474; A61M 5/50; A61M 5/31513

(57) ABSTRACT

A piercing mechanism for piercing a cartridge (10) comprises a cartridge container (20) adapted to receive and hold a cartridge (10) and allow a movement of the cartridge (10) from a start position via a piercing state to an end position, the cartridge (10) comprising a septum (14) covering a dispensing port (15) and a piston (12) adapted to be moved from a proximal position along an axial direction of the cartridge (10) to a distal position, a hollow needle (30) having a distal end (31) and a proximal end (32), the hollow needle (30) being immovably fixed in the piercing mechanism and adapted to pierce the septum (14) with the proximal end (32) when the cartridge (10) is moved from the start position via the piercing state to the end position, a plunger (40) adapted to cause the movement of the piston (12) when the plunger (40) is moved from a first position to a second position, wherein the second position is closer to the hollow needle (30) than the first position, and a spring mechanism (50) adapted to be compressed by the movement of the cartridge (10) from the start position to the end position, wherein the spring mechanism (50) protrudes with regard to the proximal end (32) of the hollow needle (30) when the plunger (40) is in the first position.

The invention relates to a piercing mechanism for piercing a cartridge. Furthermore, the invention relates to a cartridge piercing system comprising a piercing mechanism and a cartridge.

Known cartridges for storing fluids, for example, drug storage cartridges or ink storage cartridges, comprise a septum covering a dispensing port and a piston that can be moved from a proximal position along an axial direction of the cartridge to a distal position. After the septum is pierced by a needle, movement of the piston towards the distal position forces the fluid through the dispensing port out of the cartridge.

Problems may arise if the septum is pierced by the needle, however, the fluid is not immediately forced through the dispensing port out of the cartridge. In this case, the fluid may dry-out and the dispensing port and/or a fluid flow path connecting the dispensing port and the outside may get clogged.

WO 2012/022810 concerns an injection device comprising a needle unit having a front needle for penetrating the skin of a subject and a rear needle for piercing a cartridge septum of a medicament cartridge, a slideably arranged piston which is driveable towards the cartridge septum, a piston driver for moving the piston of the cartridge, a holding mechanism for releasably maintaining the cartridge in the initial position relative to the needle unit, and a shock absorber which slows down the movement between the cartridge and the needle unit. WO 2012/022810 further concerns a damping mechanism comprising a flexible element that is provided between the piston driver and an inner wall, and which is adapted to reduce the speed of movement of the cartridge relative to the needle unit. In addition to frictional damping, WO 2012/022810 proposes pneumatic damping and hydraulic damping.

However, frictional damping mechanisms may not precisely enough reduce the speed of the movement of the cartridge relative to the needle unit. Moreover, pneumatic and hydraulic damping mechanisms are rather complex and space-consuming so that such damping mechanisms are not suitable to be employed in small handheld devices, for example, in an injector device.

The invention is directed at the object of providing a piercing mechanism for piercing a cartridge which causes an improved piercing of a cartridge septum and an improved dispensing of a fluid stored in the cartridge. Furthermore, the invention is directed at the object of providing a cartridge piercing system which causes an improved piercing of a cartridge septum and an improved dispensing of a fluid stored in the cartridge.

This object is addressed by a piercing mechanism as defined in claim 1, and a cartridge piercing system as defined in claim 14.

The piercing mechanism for piercing a cartridge comprises a cartridge container adapted to receive and hold a cartridge and allow a movement of the cartridge from a start position via a piercing state to an end position, a hollow needle having a distal end and a proximal end, the hollow needle being immovably fixed in the piercing mechanism and adapted to pierce a septum of the cartridge with the proximal end when the cartridge is moved from the start position via the piercing state to the end position, a plunger adapted to cause a movement of a piston in the cartridge from a proximal position to a distal position when the plunger is moved from a first position to a second position, wherein the second position is closer to the hollow needle than the first position, and a spring mechanism adapted to be compressed by the movement of the cartridge from the start position to the end position, wherein the spring mechanism is adapted to protrude with regard to the proximal end of the hollow needle when the plunger is in the first position.

The terms “proximal” and “distal” in the sense of the present disclosure relate to the movement direction of the plunger, the piston and the cartridge. In particular, the plunger, the piston and the cartridge are moved from proximal positions in a distal direction towards distal positions.

The cartridge may be any kind of container that is configured to store a fluid, for example, a drug storage container for storing a liquid drug, and comprises a septum covering a dispensing port and a piston adapted to be moved from a proximal position along an axial direction of the cartridge to a distal position, and a hollow needle having a distal end and a proximal end. The term “drug” is meant to encompass any medicament-containing flowable drug configured to be passed through the hollow needle in a controlled manner, such as a liquid, solution, gel or fine suspension. The cartridge may, e.g., have a cylindrical shape with an opening at its proximal end, through which the piston is accessible.

The distal position of the piston is a position that is distal relative to the proximal position of the piston in the direction of the movement of the piston. In particular, the proximal position is a default position of the piston before usage of the cartridge and the distal position is a position of the piston after it has been moved from the proximal position in the distal direction of the cartridge. The distal position may be a maximum distal position of the piston or any position of the piston between the proximal position and the maximum distal position.

The cartridge container may be any kind of container means that allows a loading and a holding of a cartridge, and a subsequent movement of the cartridge in the distal direction from the initial start position via the piercing state to the final end position. Preferably, the cartridge container is fixed such in the piercing mechanism that it cannot be moved in the distal direction and the proximal direction. The piercing state is a transient location of the cartridge when its septum gets pierced by the proximal end of the hollow needle. For allowing a movement of the cartridge within the cartridge container, the cartridge container may comprise a guiding member, for example, a tubular member, that matches the shape of the cartridge and allows a sliding of the cartridge within the cartridge container from the start position to the end position.

The hollow needle may be in any kind of piercing needle, for example, a needle made of steel, which comprises at its proximal end, i.e., at the end that is facing in the proximal direction, a sharp needle tip that is adapted to pierce the septum of the cartridge, preferably at an angle of approximately 90 degrees and preferably at a center of the septum. Specifically, the hollow needle is fixed in the piercing mechanism such that it cannot move relative to the cartridge container and the cartridge.

The plunger may be any kind of moving member, e.g., a plunger rod, that is adapted to be moved by an actuator from the first position to the more distal second position and cause a movement of the piston of the cartridge in the distal direction. For example, the plunger may be adapted to be inserted through the proximal end of the cartridge into the cartridge and push the piston in the distal direction.

Before the start of the movement of the cartridge in the distal direction, the spring mechanism is either uncompressed or only partially compressed, and the spring mechanism ensures that the proximal end of the needle does not come into contact with the septum of the cartridge. When the cartridge is moved from the start position via the piercing state to the end position, the distal end of the cartridge causes a compression of the spring mechanism, for example, the distal end of the cartridge comes into contact with the proximal end of the spring mechanism and compresses the spring mechanism. Thus, the spring mechanism damps the movement of the cartridge in the distal direction. In particular, the spring mechanism may damp the movement of the cartridge in the distal direction immediately before, during, and after the piercing of the septum with the hollow needle. Therefore, a controlled and reliable piercing of the septum by the proximal end of the hollow needle, and a controlled and reliable dispensing of a fluid stored in the cartridge can be provided. Accordingly, the spring mechanism provides a double function of ensuring, before the septum piercing process has started, that the proximal end of the needle does not come into contact with the septum of the cartridge, and, after the septum piercing process has started, damping the movement of the cartridge in the distal direction.

The spring mechanism may comprise at least one spring that is adapted to store and release mechanical energy. Preferably, the spring mechanism comprises at least one coil spring which at least partially surrounds the hollow needle, wherein the distal end of the at least one coil spring is immovably fixed in the piercing mechanism. Accordingly, when the cartridge is moved from the start position via the piercing to the end position, the distal end of the cartridge may cause a compression of the coil spring in the distal direction, thereby ensuring a controlled and reliable piercing of the septum. Preferably, the hollow needle, the spring mechanism, the cartridge container and the plunger lie on the same longitudinal axis. Further preferably, the septum extends in a direction that is perpendicular to this longitudinal axis.

To ensure a controlled ending of the septum piercing process, the end position of the cartridge movement may be defined by a maximum compression of the spring mechanism. The maximum compression of the spring mechanism means a maximum travel of the spring mechanism considering a solid height of the spring mechanism, in case of a coil spring, a coil bind height where all the coils of the spring are touching, or a safe travel distance which the spring mechanism can travel safely without any permanent set caused by fatigue and stress. Additionally or alternatively, the piercing mechanism may comprise a stopping member which is adapted to stop the movement of the cartridge at the end position. For example, the stopping member may be an element that is located at an end position in the movement path of the cartridge such that an abutment of the distal end of the cartridge at the stopping member stops the movement of the cartridge. The stopping member may, e.g., be a part of the cartridge container.

Preferably, the plunger is adapted to cause a movement of the piston from the proximal position in the distal direction thereby causing a movement of the cartridge from the start position via the piercing state to the end position and a compression of the spring mechanism. Since a fluid is stored in the cartridge, before the septum of the cartridge is pierced by the hollow needle, the movement of the piston in the distal direction causes a force on the entire cartridge in the distal direction. Thus, the cartridge is caused to be moved within the cartridge container to via piercing state. However, due to the compression of the spring caused by the movement of the cartridge in the distal direction, the movement of the cartridge is damped so that a controlled and reliable piercing of the septum by the hollow needle is provided. Thus, before the septum is pierced by the hollow needle, the movement of the piston in the distal direction causes a movement of the piston together with the cartridge in the distal direction.

Further preferably, after the cartridge has reached the piercing state and the hollow needle has started to pierce the septum, the plunger is adapted to cause a further movement of the piston to the distal position, thereby causing a movement of the cartridge via the piercing state to the end position and a further compression of the spring mechanism. To explain in more detail, after the hollow needle has pierced the septum, further movement of the piston in the distal direction causes a release of the fluid that is stored in the cartridge through the dispensing port and the pierced septum into the hollow needle.

To allow a piercing of the septum by the hollow needle and a damping of the movement of the cartridge in the distal direction, in a preferred embodiment, the proximal end of the hollow needle is adapted to protrude from the spring mechanism when the plunger is in the second position

In a preferred embodiment, the piercing mechanism comprises a connecting tube having two ends, wherein the connecting tube is connected at one of its ends to the distal end of the hollow needle. The connecting tube is adapted to provide a fluid path between the hollow needle and a skin needle, i.e., a needle that may be used for injecting the fluid from the cartridge into a human or animal body. The connecting tube may be one tube element or a tube system that comprises a plurality of tube elements. Since the septum is only pierced immediately before, or preferably, simultaneously with the injection, the dispensing port of the cartridge and the connecting tube can be prevented from getting clogged.

Furthermore, since the septum can be pierced immediately before or simultaneously with an injection, in order to enable a user to change the cartridge until the moment just before the injection, the cartridge container may be adapted to be moved between a load position at which the cartridge can be loaded into the cartridge container and a closed position at which no loading or unloading of the cartridge into and from the cartridge container is possible.

The piercing mechanism may further comprise determining means adapted to determine at least one of whether a correct cartridge has been loaded into the cartridge container and whether the cartridge container is in the closed position without having a cartridge loaded therein. The determining means may be any kind of sensor, e.g., a radio frequency identification, RFID, reader, which is adapted to sense an information element, e.g. an RFID tag, which is provided on the cartridge. Alternatively, the determining means may comprise a switch that is activated when the cartridge is loaded into the cartridge container. The determining means may, e.g., determine whether the fluid stored in the cartridge has expired or whether a wrong fluid that does not match a medical indication is stored in the cartridge. Thus, a user can load a correct cartridge into the cartridge container before a piercing of the septum has taken place, e.g., the piercing of the septum has been triggered by means of operation mistakes by the user.

Preferably, the piercing mechanism comprises a lock mechanism adapted to prevent the cartridge container from being moved from the closed position to the load position. The determining means may be adapted to control the lock mechanism. For example, when the determining means have determined that a correct cartridge has been loaded into the cartridge container and the cartridge container is in the closed position, the determining means may cause the lock mechanism to prevent the cartridge container from being moved to the load position.

In another preferred embodiment, the determining means are adapted to control the lock mechanism to permanently prevent the cartridge container from being moved from the closed position to the load position. For example, after it has been determined that a correct cartridge has been loaded into the cartridge container, the determining means may control the lock mechanism to permanently prevent the cartridge container from being moved from the closed position to the load position. Preferably, the permanent lock may be caused after the movement of the cartridge from the start position towards the end position has started.

In order to facilitate a loading of the cartridge into the cartridge container, the cartridge container may comprise at its distal end a hinge element having a pin and a torsion spring, wherein the torsion spring is adapted to cause a rotation of the cartridge container around the pin from the closed position to the load position. Thus, the lock mechanism may prevent the cartridge container from being rotated around the pin.

In another preferred embodiment, the cartridge container is adapted to be moved between the load position at which the cartridge can be loaded into the cartridge container and the closed position at which no loading or unloading of the cartridge into and from the cartridge container is possible, wherein the spring mechanism is adapted to be uncompressed or compressed less than 50% of its maximum compression when the cartridge container is in the closed position and the cartridge is held by the cartridge container in the start position, or when the cartridge container is in the closed position and no cartridge is held by the cartridge container. In this case, the compression or the full compression of the spring mechanism, i.e., the damping of the cartridge movement, only starts after the cartridge is moved from the start position towards the end position. Moreover, the spring mechanism ensures that a safety distance between the proximal end of the hollow needle and the septum is kept before the cartridge is moved from the start position in the distal direction.

To provide a controlled movement of the plunger, the piercing mechanism may comprise a motor adapted to cause the movement of the plunger from the first position to the second position. Preferably, the motor is a direct current (DC) electric motor. Further preferably, the motor is adapted to provide a relatively high torque. The speed of the motor may be controlled via a microcontroller, preferably, based on a pulse-width modulation (PWM) control scheme. Further preferably, the movement of the plunger from the first position to the second position is a continuous and uninterrupted movement.

To provide a space-saving piercing mechanism, e.g., in order to be able to provide an actuator for moving the plunger at a location close to the spring mechanism, the piercing mechanism may comprise a spring guide having a curved shape, and a slinky spring, i.e., a coil spring, adapted to be moved in the spring guide, wherein the plunger is provided at one end of the slinky spring. For example, the spring guide may be substantially U-shaped such that a force that is applied by the actuator to one end of the slinky spring in a first direction causes a force at the other end of the slinky spring in a second direction that is opposite to the first direction. Specifically, the plunger may be realized as a spring head that is connected to the distal end of the slinky spring such that the spring head may be inserted through the proximal end of cartridge in order to cause a movement of the piston in the distal direction.

The invention further concerns a cartridge piercing system comprising a piercing mechanism and a cartridge. Preferably, the cartridge comprises a septum covering a dispensing port and a piston adapted to be moved from a proximal position along an axial direction of the cartridge to a distal position.

In a preferred embodiment, the cartridge piercing system comprises a skin needle adapted to be injected into a human or animal body and an extraction mechanism adapted to cause an extraction of the skin needle from the cartridge piercing system, wherein the piercing mechanism is adapted to cause the piercing of the septum with the proximal end of the hollow needle after the skin needle has been caused to be extracted from the cartridge piercing system or simultaneously with the extraction of the skin needle from the cartridge piercing system. Thus, the hollow needle can pierce the septum immediately before or approximately simultaneously with the extraction of the skin needle into the human or animal body. For this, the extraction mechanism may comprise at least one extraction spring.

The invention further concerns an injector device, in particular, an auto-injector device, which comprises a cartridge piercing system. For example, the injector device may be adapted to inject a medical drug that is stored in a cartridge into a human or animal body.

Preferred embodiments of the invention will now be described in greater detail with reference to the appended schematic drawings, wherein

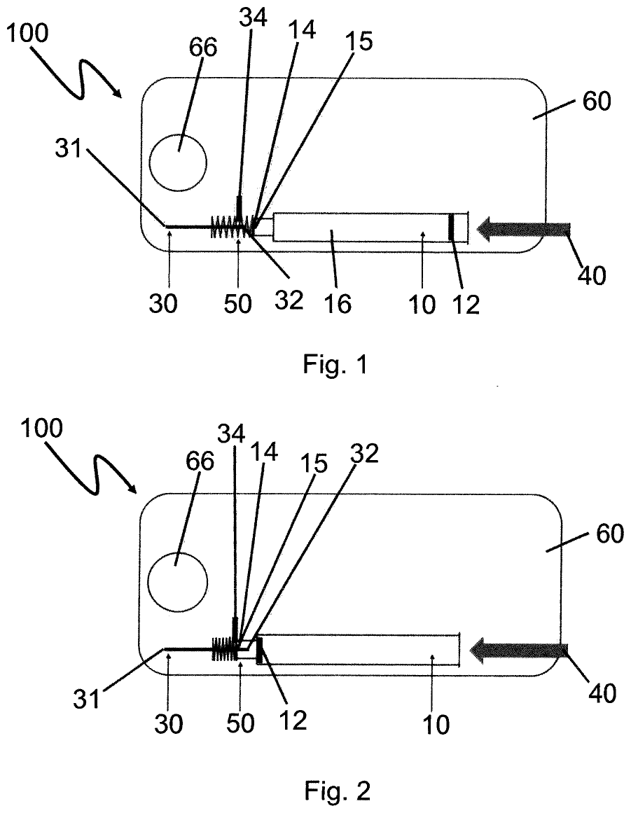

FIGS. 1 and 2 schematically show a cartridge piercing system comprising a piercing mechanism according to a first embodiment;

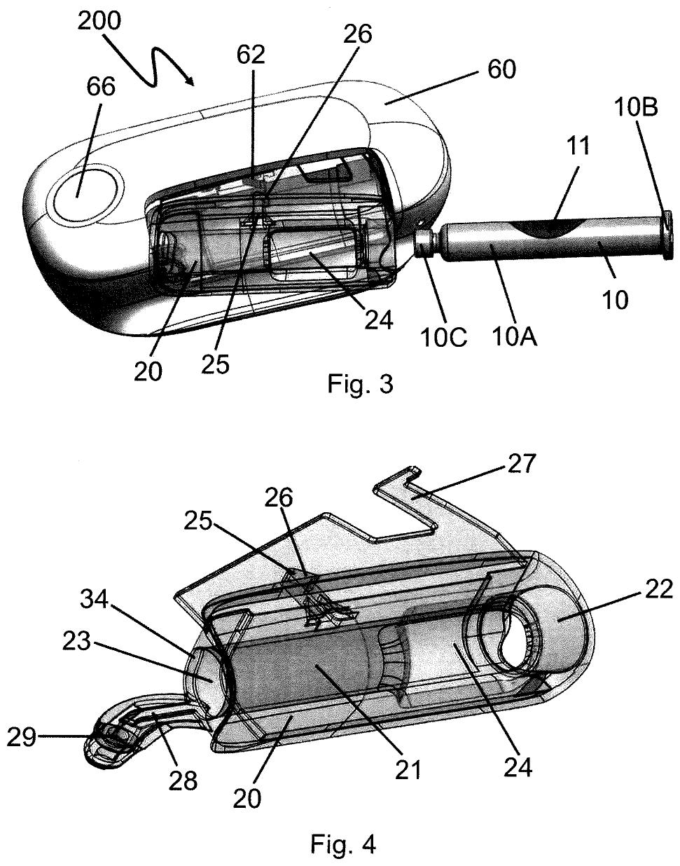

FIG. 3 shows a perspective view of an injector device with a cartridge and a cartridge container according to a second embodiment;

FIG. 4 shows a perspective view of a cartridge container according to the second embodiment;

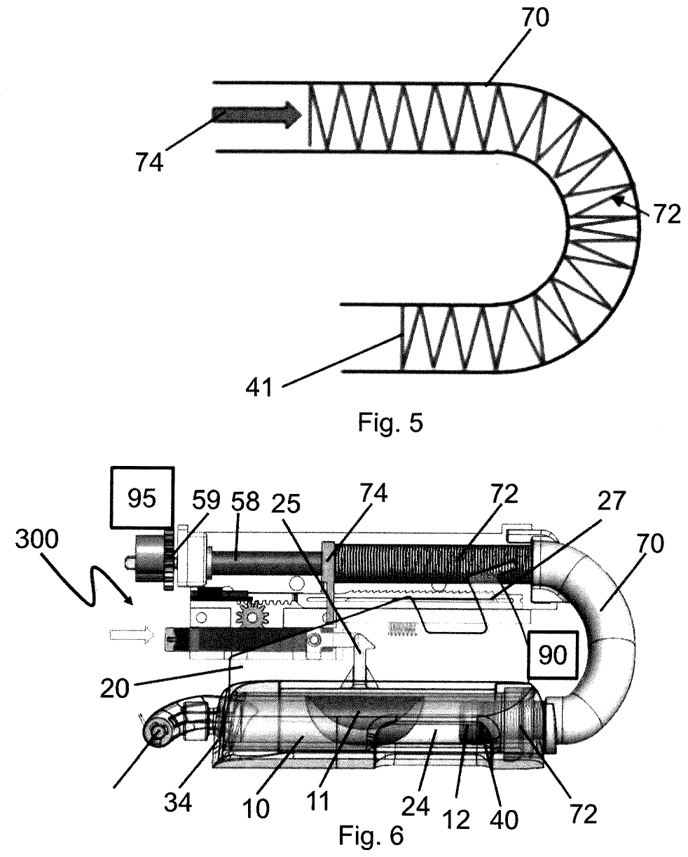

FIG. 5 schematically shows a push mechanism for pushing the piston of the cartridge;

FIGS. 6 to 9 schematically show a cartridge piercing system with a piercing mechanism according to a third embodiment;

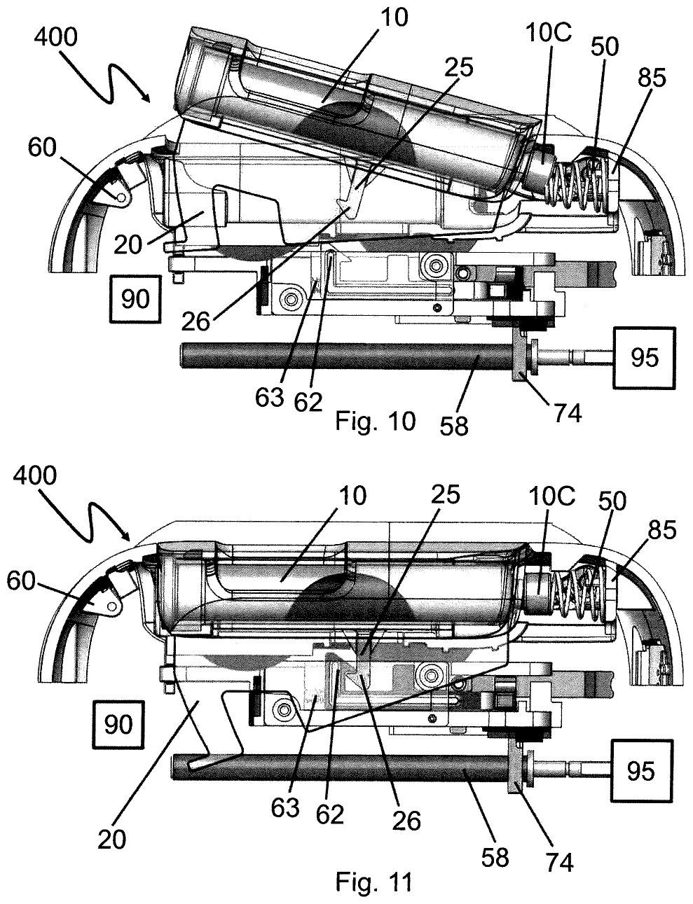

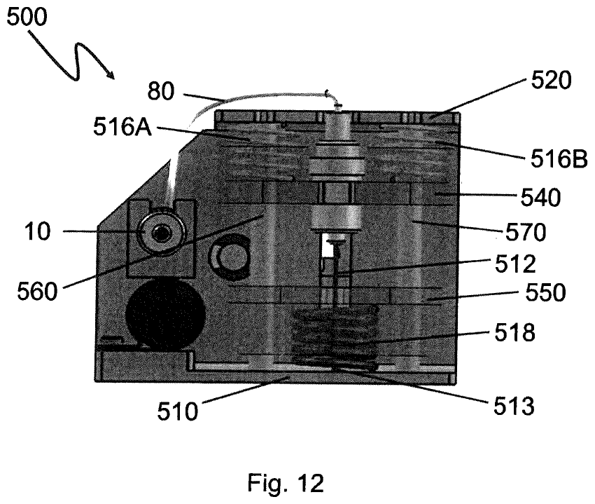

FIGS. 10 and 11 schematically show a piercing mechanism with a cartridge container and a lock mechanism according to a fourth embodiment; and

FIG. 12 schematically shows a needle ejection and retraction mechanism comprising a cartridge piercing system according to a fifth embodiment.

FIGS. 1 and 2 schematically show a cartridge piercing system 100 comprising a piercing mechanism according to a first embodiment.

The cartridge piercing system 100 according to the first embodiment comprises a cartridge 10, a cartridge container 20 (not shown in FIGS. 1 and 2), a hollow needle 30, a plunger rod 40 (schematically illustrated in FIGS. 1 and 2 by an arrow), and a coil spring 50. FIGS. 1 and 2 further schematically illustrate a housing 60, e.g., the housing of an injector device.

The cartridge 10 is a drug storage cartridge configured to store a drug 16. The cartridge 10 comprises a piston 12 that is movable from the proximal position shown in FIG. 1 to the distal position shown in FIG. 2. At its distal end, the cartridge 10 comprises a septum 14 which covers a fluid dispensing port 15. Upon movement of the piston 12 from the proximal position in the distal direction towards the distal position, in case the dispensing port 15 is opened, the drug 16 is distally forced out of the dispensing port 15. However, in case the dispensing port 15 is closed by the septum 14 and the drug 16 is stored in the cartridge 10, movement of the piston 12 from the proximal position in the distal direction towards the distal position causes a force on the entire cartridge 10 in the distal direction. FIGS. 1 and 2 further show a stopping member 34 adapted to stop the movement of the cartridge 10 in the distal direction at the end position.

A cartridge container 20 that may be employed in the first embodiment is shown in FIGS. 3 and 4 as a second embodiment. The same reference numbers in FIGS. 1 to 4 concern the same elements. FIG. 3 shows a perspective view of an injector device 200 comprising a cartridge container 20 in a load position at which the cartridge 10 can be loaded into the cartridge container 20. FIG. 4 shows a perspective view of the cartridge container 20 according to FIG. 3 alone. The cartridge container 20 can only be moved between the load position shown in FIG. 3 and a closed position, as, e.g., shown in FIGS. 6 to 11. In the closed position, the cartridge container 20 cannot be moved in the distal direction or the proximal direction. The cartridge container 20 is configured to hold the cartridge 10. When the cartridge 10 is loaded in the cartridge container 20 and the cartridge container 20 is moved to the closed position, the cartridge 10 can be moved distally from an initial start position via a piercing state to an end position. FIGS. 1, 2 and 3 further show an initiation button 66 adapted to by pushed by a user, whereupon the operation of the injector device 100, 200 is started.

The cartridge 10 shown in FIG. 3 has a substantially cylindrical shape and comprises a cylindrical main part 10A, a cylindrical proximal part 10B having a larger diameter than the main part 10A, and a cylindrical distal part 10C having a smaller diameter than the main part 10A. On the main part 10A, an information tag 11, e.g., an RFID tag, is provided.

The cartridge container 20 of the second embodiment according to FIGS. 3 and 4 comprises a substantially tubular shaped tube member 21 with a proximal opening 22 and a distal opening 23. The tube member 21 has a size and shape that fits the cartridge 10, i.e., a size to firmly hold the main part 10A of the cartridge 10, however, to still allow the cartridge 10 to be moved in the distal direction when a pushing force is applied to the proximal end of the cylindrical proximal part 10B. At least one of the proximal end 22 and the distal end 23 has a diameter adapted to stop the movement of the cartridge 10 at the end position. For example, the proximal end 22 of the cartridge container 20 may have a smaller diameter than the proximal part 10B of the cartridge 10, or the distal end 23 of the cartridge container 20 may have a smaller diameter than the main part 10A of the cartridge 10. In particular, the edge 34 of the opening 23 may act as a stopping member to stop the movement of the cartridge 10 in the distal direction at the end position. Alternatively, a maximum compression of the spring 50 or a compression of the spring to a certain extent may stop the movement of the cartridge 10 at the end position (see FIG. 2). The cartridge container 20 further comprises a window 24, a lock member 25, a guide element 27, and a hinge element 28. The window is an opening through which the information tag 11 can be inspected and the cartridge 10 can be rotated by a user from the outside. The lock member 25 comprises a first hook element 26 that is configured to be engaged with a counterpart hook element 62, which is provided in the piercing mechanism. When the cartridge container 20 is in the closed position, the engagement of the first hook element 26 and the second hook element 62 prevents the cartridge container 20 from being moved to the load position. The guide element 27 guides the movement of the cartridge container 20 between the closed position and the load position. The hinge element 28 comprises a hole 29. Within the hole 29, a pin with a torsion spring (not shown in FIG. 3) may be provided. The torsion spring is adapted to cause a rotation of the cartridge container 20 from the closed position to the load position around the pin.

Now turning back to FIGS. 1 and 2, the hollow needle 30 comprises a distal end 31 and a proximal end 32, and is immovably fixed in the piercing mechanism. The proximal end 32 has a sharp tip end which is adapted to pierce the septum 14 such that the dispensing port 15 is opened. The hollow needle 30 is made of stainless steel. The cartridge 10 with the piston 12, the cartridge container 20, the hollow needle 30, the plunger rod 40, and the coil spring 50 are all arranged along the same axial line.

The coil spring 50 comprises a distal end and a proximal end, wherein the distal end is fixed in the piercing mechanism such that a movement of the cartridge 10 in the distal direction compresses the coil spring 50, as can be seen from FIG. 2. The coil spring 50 surrounds the hollow needle 30 such that before the movement of the cartridge 10 in the distal direction, the coil spring 50 keeps a distance between the proximal end 32 of the needle 30 and the septum 14. Thus, in the state before the movement of the cartridge 10 in the distal direction shown in FIG. 1, the distal end 31 of the needle 30 protrudes distally from the distal end of the coil spring 50, whereas the proximal end of the coil spring 50 protrudes proximally from the proximal end 32 of the needle 30.

When the plunger rod 40 is moved from the first position shown in FIG. 1 in the distal direction, it pushes the piston 12 in the distal direction. In particular, as long as the needle 30 has not pierced the septum 14, the movement of the piston 12 in the distal direction causes a movement of the piston 12 together with the entire cartridge 10 in the distal direction, i.e., the cartridge 10 is forced to slide within the tube member 21 of the cartridge container 20 in the distal direction via the piercing state at which the proximal end 32 of the needle 30 pierces the septum 14.

After the septum 14 has been pierced by the needle 30, which causes the drug 16 to be dispensed from the cartridge 10 through the distal end 31 of the hollow needle 30, the cartridge 10 and the piston 12 still move with approximately the same speed until the movement of the cartridge 10 is stopped by the stopping member 34. In particular, the movement of the cartridge 10 in the distal direction ends with the situation shown in FIG. 2, in which the proximal end 32 of the needle 30 has pierced the septum 14, the coil spring 50 is compressed after being pushed by the distal end of the cartridge 10, the piston 12 is located at its most distal position, the plunger rod 40 is located at its most distal position (not shown in FIG. 2), and the drug 16 has been dispensed via the dispensing port 15 through the hollow needle 30. Thus, at the end of the septum piercing process, the coil spring 50 is compressed such that the proximal end 32 of the needle 30 protrudes from the proximal end of the coil spring 50.

FIG. 5 schematically shows an embodiment of a pushing mechanism that may be used for pushing the piston 12 shown in the first embodiment according to FIGS. 1 and 2.

The pushing mechanism comprises a spring guide 70 and slinky spring 72 which is adapted to be moved in the spring guide 70. The slinky spring 72 may be made of a metal material. The spring guide 70 has a curved shape, for example a U-shape. At its proximal end, the slinky spring 72 is adapted to be pushed by a nut 74, as indicated by the arrow. The nut 74 may me driven by a motor (not shown in FIG. 5). At its distal end, the slinky spring 72 comprises a spring head 41 which is connect to or coincides with the plunger rod 40 (not shown in FIG. 5) so that the spring head 41 and/or the plunger rod 40 are adapted to push the piston 12 of the cartridge 10 shown in FIGS. 1 to 3. Thus, the push mechanism enables a turning of the direction of the pushing force, e.g., by 90 degrees. Hence, it is possible to include the cartridge piercing system in a small-sized injector device, for example, an injector device having a shape like a computer mouse with a flat surface that is adapted to be placed on a human or animal body.

FIGS. 6 to 9 schematically show a cartridge piercing system 300 with a piercing mechanism according to a third embodiment. Specifically, FIGS. 6 to 9 show a combination of the cartridge piercing systems 100 and 200 according to FIGS. 1 to 4 with the pushing mechanism according to FIG. 5. Accordingly, the same reference numbers concern the same elements, and any repeated explanation thereof is omitted.

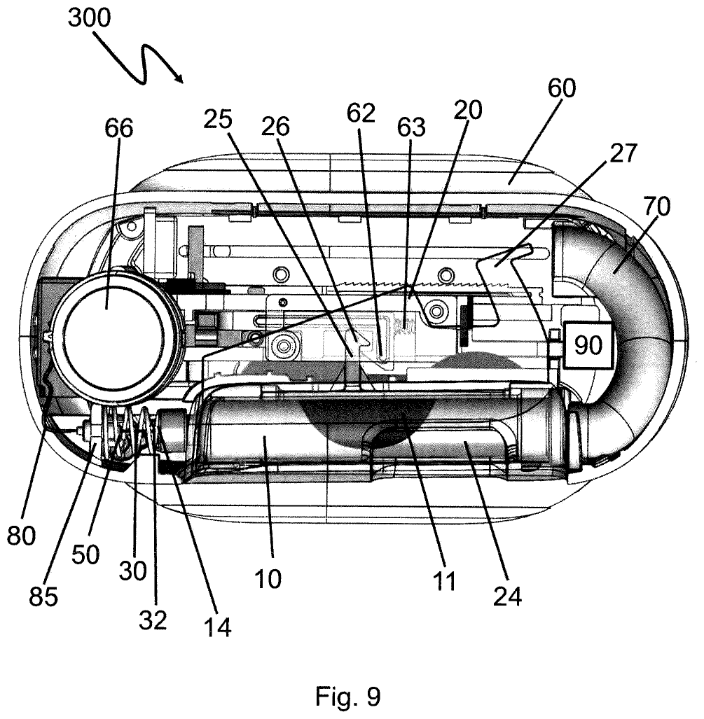

FIGS. 6 to 8 differ from FIG. 9 in that the housing 60, the needle 30 and the coil spring 50 have been omitted in FIGS. 6 to 8. Moreover, in FIGS. 6 to 8, the inside of the cartridge 10 with the piston 12 can be seen. In FIG. 9, the piercing mechanism with the needle 30 and the coil spring 50 is shown, whereas the slinky spring 72 shown in FIG. 5 has been omitted. Furthermore, FIGS. 6 to 8 schematically show a motor 95, which is not shown in FIG. 9. Moreover, for the sake of clarity, elements shown in the figures that are not relevant for the operation of the cartridge piercing system 300 have not been designated with reference numbers in FIGS. 6 to 9 and are not described in detail in the following.

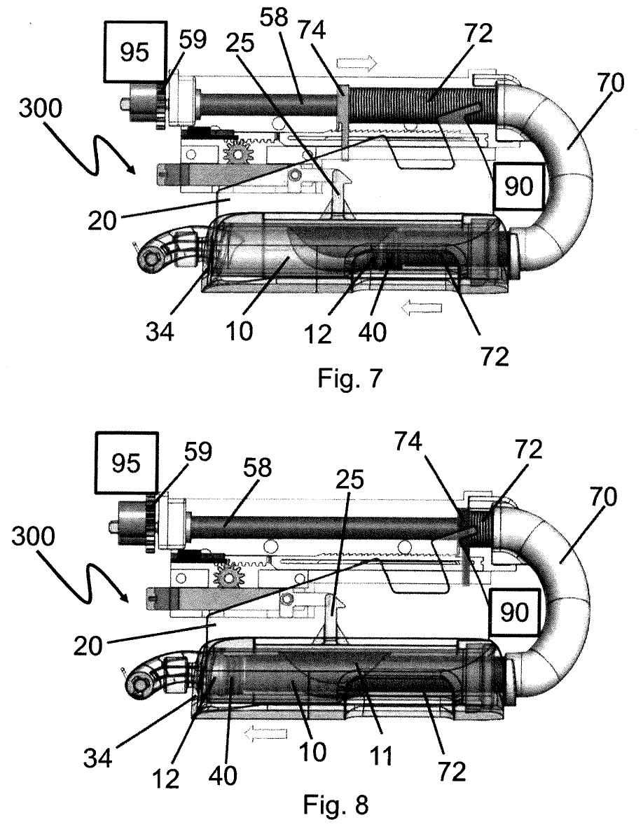

FIGS. 6 to 9 show a motor 95 which is mechanically coupled to a leadscrew 58. The leadscrew 58 comprises a driving gear 59 that can be driven, i.e., rotated, by the motor 95. The nut 74 comprises a female inner thread which fits the leadscrew 58 as a male thread so that a rotation of the driving gear 59 by the motor 95 causes a movement of the nut 74 along the leadscrew 58, as indicated by the upper arrow in FIG. 7. Thus, the nut 74 is adapted to push the slinky spring 72 through the spring guide 70. Thereby, the plunger rod 40 pushes the piston 12 in the distal direction, as indicated by the lower arrow in FIGS. 7 and 8. A rotation of the driving gear 59 by the motor 95 in the opposite direction causes a movement of the nut 74 in the opposite direction along the leadscrew 58.

When the cartridge container 20 is in the closed position and the cartridge 10 is in the start position, the piston 12 is located at its proximal position and the plunger rod 40 is located at the first position, as shown in FIG. 6. FIG. 9 shows in more detail the start position of the cartridge 10 before the plunger rod 40 has caused any movement of the piston 12 in the distal direction. As can be seen from FIG. 9, the spring 50 ensures that a safety distance between the tip end 32 of the needle 30 and the septum 14 is kept. For this, the distal end of the spring 50 is fixed to a base element 85 and the proximal end of the spring 50 is in contact with the distal end surface of the cartridge 10.

Thereafter, as shown in FIG. 7, the motor 95 rotates the driving gear 59, which causes a rotation of the leadscrew 58 and movement of the nut 74. The nut 74 pushes the proximal end of the slinky spring 72 (as indicated by the upper arrow), which pushes the slinky spring 72 through the spring guide 70. By means of this movement, the plunger rod 40 is moved from the first position in the distal direction, which pushes the piston 12 from its proximal position in the distal direction. This movement of the piston 12 initially causes a movement of the cartridge 10 within the cartridge container 20 in the distal direction so that the needle 30 pierces the septum 14 of the cartridge 10. Thus, FIG. 9 shows the piercing state of the cartridge 10. After the septum 14 has been pierced by the needle 30, the fluid 16 stored in the cartridge 10 is dispensed from the cartridge 10 through the needle 30 and through the connecting tube 80 (see FIG. 9).

FIG. 8 shows the cartridge piercing system when the plunger rod 40 has reached its end location at the second position, and the piston 12 has reached its distal end position within the cartridge 10, at which the fluid 16 has been forced out the cartridge 10.

FIGS. 6 to 9 further schematically show determining means 90 adapted to determine whether a correct cartridge 10 has been loaded into the cartridge container 20. For this, the determining means 90 comprise an RFID tag reader that is adapted to read the information stored in the RFID tag 11. Based on this information, the determining means 90 may determine whether the drug 16 stored in the cartridge 10 has expired or whether a drug 16 that does not match a medical indication is stored in the cartridge 10. Furthermore, the determining means 90 are adapted to determine whether the cartridge container 20 is in the closed position without having a cartridge 10 loaded therein.

In FIG. 9, the locking mechanism for the cartridge container 20 is shown in more detail. In the closed position of the cartridge container 20 shown FIG. 9, the first hook element 26 of the lock member 25 is engaged with the counterpart hook element 62. For enabling this engagement when moving the cartridge container 20 from the load position to the closed, the counterpart hook element 62 is provided at a spring 63, which can be compressed for allowing a movement of the counterpart hook element 62 in a direction away from the lock member 25 and thereafter the engagement of the counterpart hook element 62 with the first hook element 26.

The determining means 90 further comprise computing means (e.g., a computer unit, not shown in FIG. 9) for processing the received data and controlling means (e.g., a microcontroller, not shown in FIG. 9) for controlling the movement of the counterpart hook element 62 based on the processed data, i.e., controlling whether the counterpart hook element 62 can be moved or not. Moreover, the determining means 90 are connected to the motor 95.

FIGS. 10 and 11 schematically show a piercing mechanism 400 with a cartridge container 20 and a lock mechanism 25, 26, 62, 63 according to a fourth embodiment. The fourth embodiment is based on the pervious embodiments. Thus, the same reference numbers correspond to the same elements and any repeated explanation thereof is omitted. Contrary to the previous embodiments, in the fourth embodiment according to FIGS. 10 and 11, the needle 30, the spring guide 70 and the plunger rod 40 are not shown.

FIG. 10, similar to FIG. 3, shows the cartridge container 20 in the load position. Contrary to FIG. 3, in FIG. 10, the cartridge 10 is already loaded in the cartridge container 20. Moreover, in FIG. 10, the counterpart hook element 62 has been moved towards the spring 63, which has caused a compression of the spring 63. Thus, the cartridge container 20 can be rotated in the closed position shown in FIG. 11, in which the engagement of the counterpart hook element 62 with the first hook element 26 has taken place after a release of the spring 63. The movement of the counterpart hook element 62 in the direction towards the spring 63 has been caused by the motor 95. Accordingly, the motor 95 can also prevent a movement of the counterpart hook element 62 in the direction towards the spring 63.

Thus, the counterpart hook element 62 is adapted to prevent the cartridge container 20 from being moved from the closed position to the load position. In particular, the determining means 90 are adapted to control the counterpart hook element 62 via the motor 95. Specifically, the determining means 90 are adapted to control the counterpart hook element 62 via the motor 95 to permanently prevent the cartridge container 20 from being moved from the closed position to the load position.

After the closing of the cartridge container 20, as shown FIG. 11, the cartridge container 20 is in the closed position, and the cartridge 10 is in the start position. FIG. 12 schematically shows a needle ejection and retraction mechanism 500 comprising a cartridge piercing system according to a fifth embodiment. In particular, all elements shown in FIGS. 1 to 11 may be employed in the needle ejection and retraction mechanism 500 of FIG. 12. Thus, the same reference numbers relate to the same elements and any repetition of these elements is omitted.

The needle ejection and retraction mechanism 500 is provided in the housing of an injector have a base plate 510 and a top plate 520. Between the base plate 510 and the top plate 520, two vertical rods 560 and 570 are foreseen. The outer surface of the base plate 510 is adapted to be placed on the skin of the human or animal body. The needle ejection and retraction mechanism 500 comprises a skin needle 512 having a needle tip 513 which is adapted to pierce the skin of a human or animal body, two first springs 516A and 516B for ejecting the skin needle 512 out of the needle ejection and retraction mechanism 500, and a second spring 518 for retracting the skin needle 512 into the needle ejection and retraction mechanism 500. The two first springs 516A and 516B extend in parallel to an axis along which the skin needle 513 is ejected, and are provided between the top plate 520 and a first support element 540 which is adapted to release the spring force by the two first springs 516A and 516B on the skin needle 512. Upon ejection of the skin needle 512, the needle tip 513 protrudes from the outer surface of the base plate 510 (not shown in FIG. 12). The second spring 518 is provided between the base plate 510 and a second support element 550.

FIG. 12 further shows a drug storage cartridge 10 which is connected via a connecting tube 80 to the proximal end of the skin needle 512. Thus, the connecting tube 80 establishes a fluid path between the proximal end of the skin needle 512 and the proximal end of the hollow needle 30 (not shown in FIG. 12).

1. A piercing mechanism for piercing a cartridge, comprising
a cartridge container adapted to receive and hold a cartridge and allow a movement of the cartridge from a start position via a piercing state to an end position, the cartridge comprising a septum covering a dispensing port and a piston adapted to be moved from a proximal position along an axial direction of the cartridge to a distal position;
a hollow needle having a distal end and a proximal end, the hollow needle being immovably fixed in the piercing mechanism and adapted to pierce the septum with the proximal end when the cartridge is moved from the start position via the piercing state to the end position;
a plunger adapted to cause the movement of the piston when the plunger is moved from a first position to a second position, wherein the second position is closer to the hollow needle than the first position; and
a spring mechanism adapted to be compressed by the movement of the cartridge from the start position to the end position, wherein the spring mechanism is adapted to protrude with regard to the proximal end of the hollow needle when the plunger is in the first position.

2. The piercing mechanism according to claim 1, wherein
the spring mechanism comprises a coil spring which at least partially surrounds the hollow needle, wherein a distal end of the coil spring is immovably fixed in the piercing mechanism.

3. The piercing mechanism according to claim 1, wherein
the end position of the cartridge is defined by a maximum compression of the spring mechanism, and/or
the piercing mechanism comprises a stopping member which is adapted to stop the movement of the cartridge at the end position.

4. The piercing mechanism according to claim 1, wherein
the plunger is adapted to cause a movement of the piston from the proximal position in a distal direction thereby causing a movement of the cartridge from the start position to the end position and a compression of the spring mechanism.

5. The piercing mechanism according to claim 4, wherein
the plunger is adapted, after the cartridge has reached the piercing state, to cause a further movement of the piston to the distal position, thereby causing a movement of the cartridge via the piercing state to the end position and a further compression of the spring mechanism.

6. The piercing mechanism according to claim 1, wherein the proximal end of the hollow needle is adapted to protrude from the spring mechanism when the plunger is in the second position.

7. The piercing mechanism according to claim 1, further comprising
a connecting tube having two ends, wherein the connecting tube is connected at one of its ends to the distal end of the hollow needle, the connecting tube being adapted to provide a fluid path between the hollow needle and a skin needle.

8. The piercing mechanism according to claim 1, wherein
the cartridge container is adapted to be moved between a load position at which the cartridge can be loaded into the holding mechanism and a closed position at which no loading or unloading of the cartridge into and from the cartridge container is possible, wherein the piercing mechanism further comprises
determining means adapted to determine at least one of whether a correct cartridge has been loaded into the cartridge container and whether the cartridge container is in the closed position without having a cartridge loaded therein.

9. The piercing mechanism according to claim 8, further comprising
a lock mechanism adapted to prevent the cartridge container from being moved from the closed position to the load position, wherein
the determining means are adapted to control the lock mechanism, wherein optionally,
the determining means are adapted to control the lock mechanism to permanently prevent the cartridge container from being moved from the closed position to the load position.

10. The piercing mechanism according to claim 8, wherein
the cartridge container comprises at its distal end a hinge element having a pin and a torsion spring, wherein the torsion spring is adapted to cause a rotation of the cartridge container around the pin from the closed position to the load position.

11. The piercing mechanism according to claim 1, wherein
the cartridge container is adapted to be moved between a load position at which the cartridge can be loaded into the cartridge container and a closed position at which no loading or unloading of the cartridge into and from the cartridge container is possible; and
the spring mechanism is adapted to be uncompressed or compressed less than 50% of its maximum compression when the cartridge container is in the closed position and the cartridge is held by the holding mechanism in the start position, or when the cartridge container is in the closed position and no cartridge is held by the cartridge container

12. The piercing mechanism according to claim 1, further comprising
a motor adapted to cause the movement of the plunger from the first position to the second position.

13. The piercing mechanism according to claim 1, further comprising
a spring guide having a curved shape; and
a slinky spring adapted to be moved in the spring guide, wherein
the plunger is provided at one end of the slinky spring.

14. A cartridge piercing system, comprising
the piercing mechanism according to claim 1; and
a cartridge.

15. The cartridge piercing system according to claim 14, comprising
a skin needle adapted to be injected into a human or animal body; and
an extraction mechanism adapted to cause an extraction of the skin needle from the cartridge piercing system, wherein
the piercing mechanism is adapted to cause the piercing of the septum with the proximal end of the hollow needle after the skin needle has been caused to be extracted from the cartridge piercing system or simultaneously with the extraction of the skin needle from the cartridge piercing system.
